(19) United States
(12) Patent Application Publication
(10) Pub. No.: US 2025/0305011 A1
(43) Pub. Date: Oct. 2, 2025

(54) MICROBIAL OILS WITH HIGH LEVELS OF OMEGA-3 FATTY ACIDS

(71) Applicant: MARA Renewables Corporation, Dartmouth (CA)

(72) Inventors: Joshua Lowrey, Dartmouth (CA); Zhiyong Sun, Dartmouth (CA); Roberto E. Armenta, Dartmouth (CA); Denise Muise, Dartmouth (CA)

(21) Appl. No.: 19/232,552

(22) Filed: Jun. 9, 2025

Related U.S. Application Data
(62) Division of application No. 17/221,810, filed on Apr. 4, 2021, now Pat. No. 12,359,230.
(60) Provisional application No. 63/005,054, filed on Apr. 3, 2020.

(51) Int. Cl. C12P 7/6434; C12P 7/6427

(52) U.S. Cl. CPC C12P 7/6434; C12P 7/6427

(57) ABSTRACT

Provided herein are microbial oils and methods of making and using microbial oils with high levels of omega-3 fatty acids. Specifically, provided is a microbial oil comprising at least 85% total fatty acids, wherein the total fatty acids comprise at least 50% DHA. Also provided is a method of making a biomass comprising culturing an oil-producing microorganism in a culture medium comprising a fatty acid synthesis inhibitor, wherein the biomass comprises at least 500 mg/g oil.

### CROSS-REFERENCE TO RELATED APPLICATIONS

This application is a divisional of U.S. Non Provisional application Ser. No. 17/221,810, filed Apr. 4, 2021, which claims priority to U.S. Provisional Application No. 63/005,054, filed Apr. 3, 2020, which are incorporated by reference herein in their entirety.

### BACKGROUND

Oil from microorganisms is produced as a result of two parallel fatty acid synthesis pathways: the classical fatty acid synthesis (FAS) pathway and the polyunsaturated fatty acid (PUFA) synthase pathway. Medium chain fatty acids like myristic (C14:0) and palmitic acid (C16:0) are generally produced from the FAS pathway and long chain polyunsaturated fatty acids (LC-PUFA) like docosahexaenoic acid (DHA, C22:6 n-3) and docosapentaenoic acid (DPA, C22:5 n-6) are generally produced from the PUFA synthase pathway. The resultant fatty acid profile, however, varies greatly across microorganisms, depending on the relative activity of these parallel pathways.

### BRIEF SUMMARY

Provided herein are microbial oils and methods of making and using microbial oils with high levels of omega-3 fatty acids. Specifically, provided is a microbial oil comprising at least 85% total fatty acids by weight, wherein the total fatty acids comprise at least 50% DHA by weight. Also provided is a method of making a biomass comprising culturing an oil-producing microorganism (e.g., Aurantiochytrium) in a culture medium comprising a fatty acid synthesis inhibitor, wherein the biomass comprises at least 500 mg/g oil.

### BRIEF DESCRIPTION OF THE DRAWINGS

FIG. 1 is a graph showing fatty acid profiles of Aurantiochytrium sp. (G3) utilizing different initial fatty acid synthesis inhibitor dosing timepoints at 20° C.

FIG. 2 is a graph showing fatty acid profiles of Aurantiochytrium sp. (G3) in varying concentrations of fatty acid synthesis inhibitor at 20° C.

FIG. 3 is a graph showing fatty acid profiles of Aurantiochytrium sp. (G3) in various fatty acid synthesis inhibitor dosing strategies at 20° C. Treatments indicating 3× and 6× are multiple additions of fatty acid synthesis inhibitor at equally-spaced time intervals starting at 24 hours after inoculation. For the 3× experiment, the interval was every 24 hours. For the 6× experiment, the interval was every 12 hours.

FIG. 4 is a graph showing final product (DHA, total fatty acid (TFA), and saturated fatty acid (SFA)) concentration in Aurantiochytrium sp. (G3) in various fatty acid synthesis inhibitor dosing strategies at 20° C. Treatments indicating 3× and 6× are multiple additions of fatty acid synthesis inhibitor at equally-spaced time intervals (every 12 hours) starting 24 hours after inoculation.

FIG. 5 is a graph showing yield of product (SFA and DHA) per unit of glucose consumed in Aurantiochytrium sp. (G3) in various fatty acid synthesis inhibitor dosing strategies at 20° C. Treatments indicating 3× and 6× are multiple additions of fatty acid synthesis inhibitor at equally-spaced time intervals (every 12 hours) starting at 24 hours after inoculation.

FIG. 6 is a graph showing fatty acid profiles of Aurantiochytrium sp. (G3) from 25° C. and 20° C. cultivation conditions at flask scale.

FIG. 7 is a graph showing the fatty acid profiles of Aurantiochytrium sp. (G3). Controls were cultured in flask at 25° C. with no chemical inhibitor. The full-length fermentation runs were fed-batch fermentations in 30 L fermentor at the noted temperature, and the fatty acid synthesis inhibitor treatment was cultured at 20° C. with a pulsed addition of 12 μM total cerulenin at flask scale.

### DETAILED DESCRIPTION

Omega-3 fatty acids, including docosahexaenoic acid (DHA, C22:6 n-3) and eicosapentaenoic acid (EPA, C20:5 n-3) from the long chain-polyunsaturated fatty acid (LC-PUFA) family, are essential fatty acids for humans and non-human mammals. The amount of omega-3 fatty acids required in a microbial oil varies based on applications. For example, dietary supplements and pharmaceuticals typically prefer higher concentrations of omega-3 fatty acids per unit of oil. To achieve such concentrations, the omega-3 fatty acids are typically concentrated by transesterification and molecular distillation (Bonilla-Mendez and Hoyos-Concha, Corpoica Cienc Tecnol Agropecuaria, Mosquera (Colombia), 19 (3): 645-668 (2018)). However, the final concentrated products are typically in ethyl-ester (EE) chemical form instead of the triglyceride (TG) form, the natural chemical structure when the lipids are synthesized by microorganisms. Not only does the EE form require additional processing that may compromise its olfactory characteristics, it has also been shown that the EE form of omega-3 is less bioavailable to the human body than the original TG form.

Common strategies to increase omega-3 content include selecting high omega-3 microbial strains, classical mutagenesis, and genetic modification (Lian et al., Appl Biochem Biotechnol, 162:935-941 (2010)). Alternatively, chemicals with potential to affect the fatty acid synthesis metabolism under various mechanisms have been tested in different microorganisms. However, efficient and effective methods of applying such principles to achieve a commercially meaningful increase in DHA content while avoiding or minimizing any undesirable changes in other fatty acid components, including DPA and saturated fatty acids, are lacking.

Described herein are oils containing high levels of omega-3 fatty acid with DHA accounting for approximately 50-70% of the fatty acid profile. Also described are methods using fatty acid synthesis inhibitors to suppress less-desirable products of the FAS pathway in favor of the high-value PUFA products from the PUFA synthase pathway. The provided oils contain high concentrations of DHA without sacrificing the productivity of the microorganism. Further, described are methods of using inhibitors that inhibit the fatty acid synthase multienzyme complex as described herein. The examples illustrate fatty acid profiles of G3, showing a substantial increase in DHA content (up to more than 83.3% when compared to the control) and even more with pulsed-addition of a fatty acid synthesis inhibitor. The outcome of almost 70% DHA in TG form in an oil exceeds reported DHA concentrations.

Microorganisms, including Thraustochytrids, produce oil containing a variety of lipids, including fatty acids in various forms and amounts. As used herein, the term lipid includes phospholipids, free fatty acids, esters of fatty acids, triacylglycerols, sterols and sterol esters, carotenoids, xanthophylls (e.g., oxycarotenoids), hydrocarbons, and other lipids known to one of ordinary skill in the art. Fatty acids are hydrocarbon chains that terminate in a carboxyl group, being termed unsaturated if they contain at least one carbon-carbon double bond, and polyunsaturated when they contain multiple carbon-carbon double bonds. For example, microorganisms can produce (i) short-chain fatty acids (SCFA), which are fatty acids with aliphatic tails of fewer than six carbons (e.g., butyric acid); (ii) medium-chain fatty acids (MCFA), which are fatty acids with aliphatic tails of 6-12 carbons; (iii) long-chain fatty acids (LCFA), which are fatty acids with aliphatic tails of greater than 13 carbons. Various microorganisms produce varying types and amounts of these fatty acids. Provided herein are microorganisms and methods that shift production of these fatty acids away from medium-chain fatty acids produced by the FAS pathway to long-chain fatty acids produced by the PUFA synthase pathway. Fatty acid synthesis (FAS) is defined as the creation of fatty acids from acetyl-CoA and NADPH through the action of enzymes called fatty acid synthases. The PUFA synthase pathway enables the synthesis of polyunsaturated fatty acids de novo from malonyl-CoA by large multi-domain, multi-subunit enzymes. The major end-product of the FAS pathway is palmitate, while the major end-product of the PUFA synthases are PUFAs such as DHA and DPA.

Thus, provided herein are microbial oils and methods for making and using microbial oils. The oils include fatty acids in the form of monoglycerides, diglycerides, and triglycerides, as well as free fatty acids and phospholipids. Optionally, the microbial oil comprises at least 90% (e.g., 91%, 92%, 93%, 94%, 95%, 96%, 97%, 98%, or 99%) triglycerides. Optionally, the microbial oil comprises at least 95% triglycerides.

The oils also contain at least 85% (e.g., 86%, 87%, 88%, 89%, 90%, 91%, 92%, 93%, 94%, 95%, 96%, 97%, 98%, or 99%) by weight total fatty acids (TFA). Optionally, the oil contains between 85% to 99% total fatty acids by weight. Optionally, the microbial oil comprises 85% to 95% total fatty acids by weight. Optionally, the microbial oil comprises at least 90% total fatty acids by weight.

The percentages in reference to oils or total fatty acids are recited throughout by weight percent. For example, when a microbial oil comprises at least 90% total fatty acids, the oil contains at least 90% total fatty acids by weight of the oil. Also, total fatty acids contain specific fatty acids and percentages of the specific fatty acid are expressed throughout as weight % of the total fatty acids. For example, when the total fatty acids in the oil contain DHA, the amount of DHA is expressed as weight % of the total fatty acids. For example, the total fatty acids comprise at least 50% DHA by weight.

As described, the total fatty acids of the provided oils contain DHA. Optionally, the total fatty acids comprise at least 35%, at least 40%, or at least 45% DHA. Optionally, the total fatty acids comprise at least 50% DHA. Optionally, the total fatty acids include at least 60% DHA. Optionally, the total fatty acids include 50% to 70% DHA. Optionally, the total fatty acids comprise 60% to 70% DHA.

Optionally, the oil also contains between 6% to 18% DPA. Optionally, the oil contains between 10% to 18% DPA. Optionally, the oil contains between 6% to 10% DPA. Optionally, the total fatty acids comprise at least 60% DHA and between 10% by weight to 18% DPA. Optionally, the ratio of DHA to DPA is less than or equal to 7:1 or 6:1. Optionally, the total fatty acids comprise at least 50% DHA and the ratio of DHA to DPA is less than or equal to 7:1. Optionally, the total fatty acids comprise at least 50% DHA and the ratio of DHA to DPA is less than or equal to 6:1.

Optionally, the oil contains less than 1% stearic acid. Optionally, the total fatty acids comprise 0.01% to 1% stearic acid or 0.001% to 1% by weight stearic acid.

Optionally, the microbial oil comprises at least 85% total fatty acids, wherein the total fatty acids comprise at least 50% DHA, between 6% to 18% DPA, and less than 1% stearic acid. Optionally, the microbial oil comprises at least 85% total fatty acids, wherein the total fatty acids comprise at least 60% DHA.

Optionally, the total fatty acids comprise less than 3%, 2% or 1% eicosapentaenoic acid. Optionally, the total fatty acids comprise 0.01% to 1% eicosapentaenoic acid or 0.001% to 1% eicosapentaenoic acid. Optionally, the total fatty acids comprise 0.01% to 2% eicosapentaenoic acid or 0.001% to 2% eicosapentaenoic acid. Optionally, the total fatty acids comprise 0.01% to 3% eicosapentaenoic acid or 0.001% to 3% eicosapentaenoic acid.

Optionally, the total fatty acids comprise less than 5%, less than 4%, or less than 3% pentadecanoic acid. Optionally, the total fatty acids comprise 0.01% to 3%, 0.01% to 4%, or 0.01% to 5% pentadecanoic acid.

Optionally, the total fatty acids comprise less than 45%, 40%, 35%, 30%, or 25% saturated fatty acids (SFAs). Saturated fatty acids in the oils produced by the herein described method include, but are not limited to, C12:0 (lauric acid), C14:0 (myristic acid), C15:0 (pentadecanoic acid), C16:0 (palmitic acid), C17:0 (heptadecanoic acid), and C18:0 (stearic acid). Optionally, the total fatty acids comprise between 10% and 45% saturated fatty acids (e.g., 10% and 40%, 10% and 30, 10% and 20%, 15% and 30%, 15% and 20%, 20% and 30%, or 20% and 25% saturated fatty acids).

Optionally, the total fatty acids comprise 0.001% to 2.0% (e.g., 0.01% to 2.0% or 0.05% to 2.0%) arachidonic acid (C20:4 (n-6)). Optionally, the total fatty acids comprise 0.001% to 1% (e.g., 0.01% to 1%) eicosatetraenoic acid (C20:4 (n-3)).

Optionally, the total fatty acids comprise less than 5% by weight myristic acid. Optionally, the total fatty acids comprise 0.001% to 5%, (e.g., 0.01% to 5%, 0.1% to 5%, 0.01% to 4%, 0.1% to 4% or 1% to 4%) by weight myristic acid.

Optionally, the total fatty acids comprise less than 40% by weight palmitic acid. Optionally, the total fatty acids comprise between 5% and 40%, 5% and 30%, 10% and 40%, 10% and 30%, 20% and 40%, 20% and 30%, 20% and 40%, 20% and 25%, or 25% and 40% by weight palmitic acid.

Optionally, the total fatty acids comprise 0.1% to 0.5% or 0.001% to 0.5% by weight heptadecanoic acid.

Optionally, the fatty acids in the biomass or isolated therefrom comprise less than 3% by weight eicosapentaenoic acid, less than 5% by weight pentadecanoic acid, and less than 45% saturated fatty acids.

Optionally, the fatty acids in the biomass or isolated therefrom comprise 0.001% to 2.0%, 0.01% to 2.0%, or 0.05% to 2.0% arachidonic acid, less than 5% by weight myristic acid, and less than 40% by weight palmitic acid.

Also provided are microbial biomasses comprising oil. The microbial biomass comprises between 40 to 75% total fatty acids by weight of the total biomass. Optionally, the oil in the biomass comprises DHA and the biomass comprises 20% to 55% DHA by weight of the biomass. Optionally, the oil in the biomass comprises between 2.5% to 8% DPA by weight of the biomass. Optionally, the oil contains between 4% to 8% DPA by weight of the biomass. Optionally, the oil contains between DPA 2.5% to 4% by weight of the biomass.

Eukaryotic microorganisms useful for producing the provided microbial oils and biomasses include, but are not limited to, microorganisms selected from the genus Oblongichytrium, Aurantiochytrium, Thraustochytrium, Schizochytrium, and Ulkenia or any mixture thereof. Optionally, the oil-producing eukaryotic microorganisms are microorganisms with an 18S sequence with at least 97%, 98%, 99% or 100% identity to the nucleic acid sequence set forth in SEQ ID NO:1. Optionally, the oil-producing microorganisms are microorganisms of the strain Aurantiochytrium limacinum. Optionally, the eukaryotic microorganism is the same as the microorganism deposited with the International Depositary Authority of Canada (IDAC), National Microbiology Laboratory, Public Health Agency of Canada, 1015 Arlington Street, Winnipeg, Manitoba Canada R3E 3R2, on Jul. 22, 2016, having IDAC assigned Accession No. 220716-01. This deposit will be maintained under the terms of the Budapest Treaty on the International Recognition of the Deposit of Microorganisms for the Purposes of Patent Procedure. This deposit is exemplary and was made merely as a convenience for those of skill in the art and is not an admission that a deposit is required for patentability. The terms “G3,” “G3-1” or “G3-1 strain” or “strain G3-1” are used herein interchangeably to refer to the eukaryotic microorganism having IDAC Accession No. 220716-01.

Nucleic acid, as used herein, refers to deoxyribonucleotides or ribonucleotides and polymers and complements thereof. The term includes deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs). Unless otherwise indicated, conservatively modified variants of nucleic acid sequences (e.g., degenerate codon substitutions) and complementary sequences can be used in place of a particular nucleic acid sequence recited herein. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms identical or percent identity, in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be substantially identical. This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated as appropriate. The sequence comparison algorithm then calculates the percent sequence identities for the test sequences relative to the reference sequence based on the program parameters.

A comparison window, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981); by the homology alignment algorithm of Needleman & Wunsch, J. Mol. Biol. 48:443 (1970); by the search for similarity method of Pearson & Lipman, Proc. Nat'l. Acad. Sci. USA 85:2444 (1988); by computerized implementations of these algorithms (GAP, BESTFIT, FASTA, and TFASTA in the Wisconsin Genetics Software Package, Genetics Computer Group, 575 Science Dr., Madison, WI); or by manual alignment and visual inspection (see, e.g., Current Protocols in Molecular Biology (Ausubel et al., eds. 1995 supplement)).

A preferred example of an algorithm that is suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. BLAST and BLAST 2.0 are used, with the parameters described herein, to determine percent sequence identity for nucleic acids or proteins. Software for performing BLAST analyses is publicly available through the National Center for Biotechnology Information, as known in the art. This algorithm involves first identifying high scoring sequence pairs (HSPs) by identifying short words of a selected length (W) in the query sequence, which either match or satisfy some positive-valued threshold score T when aligned with a word of the same length in a database sequence. T is referred to as the neighborhood word score threshold (Altschul et al.). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The Expectation value (E) represents the number of different alignments with scores equivalent to or better than what is expected to occur in a database search by chance. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, expectation (E) of 10, and the BLOSUM62 scoring matrix (see Henikoff & Henikoff, Proc. Natl. Acad. Sci. USA 89:10915 (1989)), alignments (B) of 50, expectation (E) of 10, M=5, N=−4, and a comparison of both strands.

Provided herein are methods of making biomass using oil-producing microorganisms. Specifically, provided is a method of making a biomass comprising culturing an oil-producing Aurantiochytrium microorganism in a culture medium comprising a fatty acid synthesis inhibitor, wherein the biomass comprises at least 350, 400, 450 or 500 mg/g oil. The biomass can be used for making an oil having the characteristics described herein. The method further comprises isolating the oil from the biomass.

The culturing step can be carried out at a temperature of 15° C. to 28° C. (e.g., 15, 16, 17, 18, 19, 20, 21, 22, 23, 24, 25, 26, 27 or 28° C.). Optionally, the culture medium is at a temperature of 18° C. to 22° C., 22° C. to 28° C., 22° C. to 25° C., or 25° C. to 28° C.

The fatty acid synthesis inhibitor can be added to the culture medium before, after or concurrently with addition of the microorganisms to the culture medium. Optionally, the fatty acid synthesis inhibitor is fed to the culture medium either continuously or intermittently. Optionally, the method further comprises adding the microorganisms to the culture medium and then adding the fatty acid synthesis inhibitor to the culture medium at least 6, 12, 24 or 48 hours after addition of the microorganisms to the culture medium. Optionally, the method further comprises adding the microorganisms to the culture medium and then adding the fatty acid synthesis inhibitor to the culture medium between 24 to 48 hours after addition of the microorganisms to the culture medium. Optionally, the method further comprises adding the microorganisms to the culture medium and then adding the fatty acid synthesis inhibitor to the culture medium between 6 to 24 hours after addition of the microorganisms to the culture medium. Optionally, the method further comprises adding the microorganisms to the culture medium and then adding the fatty acid synthesis inhibitor to the culture medium between 12 to 24 hours after addition of the microorganisms to the culture medium.

The fatty acid synthesis inhibitor can be added to the culture medium in one or more doses. Optionally, the method further comprises adding the microorganisms to the culture medium and the culturing comprises adding the fatty acid synthesis inhibitor in 1, 2, 3, 4, 5, or 6 doses, for example, beginning 6, 12, 24, or 48 hours after addition of the microorganisms to the culture medium. Optionally, the one or more doses are added every 6, 12, or 24 hours. By way of example, 3 doses of the fatty acid inhibitor can be added to the culture medium every 12 hours starting 24 hours after the microorganisms are added to the culture medium.

The total amount of fatty acid synthesis inhibitor added to the culture medium comprises between 3 μM and 40 μM. Optionally, the fatty acid synthesis inhibitor is administered in one or multiple doses (e.g., 2, 3, 4, 5, 6, 7, 8, 9, or 10 doses) amounting to a total of inhibitor between 3 μM and 40 μM. For example, the fatty acid synthesis inhibitor can be added in 6 doses of 2 μM amounting to a total of 12 μM. By way of other examples, the fatty acid synthesis inhibitor can be added 3 times at doses of 1, 2, 3, or 8 μM. Optionally, the fatty acid synthesis inhibitor can be added 6 times at doses of 0.5, 1, 2, 3, and 4 μM. Optionally, the total concentration of the fatty acid synthesis inhibitor added to the culture medium during the culturing comprises between 30 μg and 700 μg of inhibitor per gram of biomass. Optionally, the fatty acid synthesis inhibitor is administered in 1, 2, 3, 4, 5, 6, 7, 8, 9, or 10 doses amounting to between 30 μg and 700 μg per gram of biomass. By way of example, the fatty acid synthesis inhibitor can be added in 6 doses of 40 μg per gram of biomass amounting to a total of 240 μg. By way of other examples, the fatty acid synthesis inhibitor can be added 3 times at doses of approximately 12, 24, 42, and 120 μg per gram of biomass. Optionally, the fatty acid synthesis inhibitor can be added 6 times at doses of 5.5, 11, 25, 40 and 62 μg per gram of biomass.

Suitable fatty acid synthesis inhibitors include, but are not limited to fatty acid synthase inhibitors. Optionally, the fatty acid synthase inhibitor is selected from the group consisting of quercetin, α-mangostin, thiolactomycin, triclosan, isoniazid, decynoyl-N-acetylcysteamine (NAC), and cerulenin.

As described above, eukaryotic microorganisms useful for the provided methods include, but are not limited to, microorganisms selected from the genus Oblongichytrium, Aurantiochytrium, Thraustochytrium, Schizochytrium, and Ulkenia or any mixture thereof. Optionally, the oil-producing eukaryotic microorganisms are microorganisms with an 18S sequence with at least 97%, 98%, 99% or 100% identity to the sequence set forth in SEQ ID NO: 1. Optionally, the eukaryotic microorganism has IDAC Accession No. 220716-01.

As described herein, the biomass comprises fatty acids, e.g., fatty acids comprising at least at least 90%, 91%, 92%, 93%, 94%, 95%, 96%, 97%, 98%, or 99% triglycerides as described above. Oils in the biomass also contain at least 85%, total fatty acids by weight as described above.

Optionally, the fatty acids isolated from the biomass comprise at least 50% DHA by weight as described above.

The fatty acids in the biomass optionally comprise between 6% to 18% DPA by weight as described above. Optionally, the ratio of DHA to DPA in the fatty acids, the oil in the biomass, or isolated therefrom is less than or equal to 7:1 or 6:1 as described above.

The oil in the biomass or isolated therefrom optionally contains less than 1% by weight stearic acid as described above.

Optionally, the fatty acids in the biomass or isolated therefrom comprise less than 3% by weight eicosapentaenoic acid, less than 5% by weight pentadecanoic acid, and less than 45% saturated fatty acids as described above.

Optionally, the fatty acids in the biomass or isolated therefrom comprise 0.001% to 2.0% or 0.01% to 2.0% or 0.05% to 2.0%, arachidonic acid, less than 5% by weight myristic acid, and less than 40% by weight palmitic acid. Optionally, the fatty acids comprise 0.1% to 0.5% or 0.001% to 0.5% by weight heptadecanoic acid.

Culture medium as used in the described methods supplies various nutritional components, including a carbon source and a nitrogen source, for the microorganisms. Medium for culture can include any of a variety of carbon sources. Examples of carbon sources include fatty acids, lipids, glycerols, triglycerols, carbohydrates, polyols, amino sugars, and any kind of biomass or waste stream. Fatty acids include, for example, oleic acid. Carbohydrates include, but are not limited to, glucose, cellulose, hemicellulose, fructose, dextrose, xylose, lactulose, galactose, maltotriose, maltose, lactose, glycogen, gelatin, starch (corn or wheat), acetate, m-inositol (e.g., derived from corn steep liquor), galacturonic acid (e.g., derived from pectin), L-fucose (e.g., derived from galactose), gentiobiose, glucosamine, alpha-D-glucose-1-phosphate (e.g., derived from glucose), cellobiose, dextrin, alpha-cyclodextrin (e.g., derived from starch), and sucrose (e.g., from molasses). Polyols include, but are not limited to, maltitol, erythritol, and adonitol. Amino sugars include, but are not limited to, N-acetyl-D-galactosamine, N-acetyl-D-glucosamine, and N-acetyl-beta-D-mannosamine. The carbon source can be present in the heterotrophic medium at a concentration of 200 g/L, 175 g/L, 150 g/L, 100 g/L, 60 g/L or less, e.g., at a concentration of 1 to 200 g/L, 5 to 200 g/L, 10 to 200 g/L, 50 to 200 g/L, or 100 to 200 g/L.

The microorganisms can be cultured in medium having a chloride concentration from about 0.5 g/L to about 50.0 g/L (e.g., a chloride concentration from about 0.5 g/L to about 35 g/L, from about 18 g/L to about 35 g/L, or from about 2 g/L to about 35 g/L). The microorganisms described herein can be grown in low chloride conditions, e.g., from about 0.5 g/L to about 20 g/L, or from about 0.5 g/L to about 15 g/L.

The culture medium optionally includes NaCl. The culture medium can include non-chloride-containing sodium salts as a source of sodium. Examples of non-chloride sodium salts suitable for use in accordance with the present methods include, but are not limited to, soda ash (a mixture of sodium carbonate and sodium oxide), sodium carbonate, sodium bicarbonate, sodium sulfate, and mixtures thereof. See, e.g., U.S. Pat. Nos. 5,340,742 and 6,607,900, the entire contents of each of which are incorporated by reference herein. Optionally, the medium comprises 9 g/L chloride when using 20 g/L of carbon, 20 g/L soy peptone, and 5 g/L yeast extract. The medium can comprise 35 g/L chloride when the medium contains 10 g/L carbon, 5 g/L soy peptone, 5 g/L yeast extract and 10 g/L agar. The medium can comprise 2 g/L chloride when the medium contains 20-40 g/L carbon, 1 g/L yeast extract, 1-20 g/L monosodium glutamate (MSG), 0.3-2.0 g/L phosphates, 4 g/L magnesium sulfate, 5-10 g/L ammonium sulfate, 1.5 mL/L trace elements solution, 1 mL/L of vitamin B solution, and 0.1 g/L CaCl₂).

Medium for a microorganism culture can include any of a variety of nitrogen sources. Exemplary nitrogen sources include ammonium solutions (e.g., NH₄ in H₂O), ammonium or amine salts (e.g., (NH₄)₂SO₄, (NH₄)₃PO₄, NH₄NO₃, NH₄OOCH₂CH₃(NH₄Ac)), peptone, soy peptone, tryptone, yeast extract, malt extract, fish meal, sodium glutamate, soy extract, casamino acids and distiller grains. Concentrations of nitrogen sources in suitable medium typically range between and including about 1 g/L and about 25 g/L (e.g., about 5 to 20 g/L, about 10 to 15 g/L, or about 20 g/L). Optionally, the concentration of nitrogen is about 10 to 15 g/L when yeast extract is the source of complex nitrogen in the medium. Optionally, the concentration of nitrogen is about 1 to 5 g/L when soy peptone is in the medium along with L-Glutamic acid monosodium salt hydrate (MSG) or ammonium sulfate.

The medium optionally includes a phosphate, such as potassium phosphate or sodium-phosphate (e.g., potassium phosphate monobasic).

Inorganic salts and trace nutrients in the medium can include ammonium sulfate, sodium bicarbonate, sodium orthovanadate, potassium chromate, sodium molybdate, selenous acid, nickel sulfate, copper sulfate, zinc sulfate, cobalt chloride, iron chloride, manganese chloride calcium chloride, and EDTA. Optionally, the medium includes at least 1.5 ml/L of a trace element solution. Optionally, the trace element solution comprises 2 mg/mL copper (II) sulfate pentahydrate, 2 mg/mL zinc sulfate heptahydrate, 1 mg/mL cobalt (II) chloride hexahydrate, 1 mg/mL manganese (II) chloride tetrahydrate, 1 mg/mL sodium molybdate dihydrate, and 1 mg/mL nickel (II) sulfate.

The medium can include magnesium sulfate, optionally, with a trace element solution and/or potassium phosphate monobasic.

Vitamins such as pyridoxine hydrochloride, thiamine hydrochloride, calcium pantothenate, p-aminobenzoic acid, riboflavin, nicotinic acid, biotin, folic acid and vitamin B12 can be included in the culture medium.

The pH of the medium can be adjusted to between and including 3.0 and 10.0 using acid or base, where appropriate, and/or using the nitrogen source. Optionally, the medium is sterilized.

Generally a medium used for culture of a microorganism is a liquid medium. However, the medium used for culture of a microorganism can be a solid medium. In addition to carbon and nitrogen sources as discussed herein, a solid medium can contain one or more components (e.g., agar and/or agarose) that provide structural support and/or allow the medium to be in solid form.

Cultivation of the microorganisms can be carried out using known conditions, for example, those described in International Publication Nos. WO 2007/069078 and WO 2008/129358. For example, cultivation can be carried out for 1 to 30 days (e.g., 1 to 21 days, 1 to 15 days, 1 to 12 days, 1 to 9 days, or 3 to 5 days). Cultivation can be carried out at temperatures between 4 to 30° C. Optionally, cultivation is carried out by aeration-shaking culture, shaking culture, stationary culture, batch culture, fed-batch culture, continuous culture, rolling batch culture, wave culture, or the like. Optionally, cultivation is carried out with a dissolved oxygen content of the culture medium between 1 and 20%, between 1 and 10%, or between 1 and 5%.

The biomass as described herein can be incorporated into a final product (e.g., food or feed supplement, biofuel, etc.). Thus, provided is a method of using the protein-rich biomass. The method optionally includes incorporating the protein-rich biomass into a foodstuff (e.g., a pet food, a livestock feed, or an aquaculture feed).

Oils or lipids can be isolated from the described microorganism culture and used in various food and feed supplements. Suitable food or feed supplements into which the oils can be incorporated include beverages such as milk, water, sports drinks, energy drinks, teas, and juices; confections such as candies, jellies, and biscuits; fat-containing foods and beverages such as dairy products; processed food products such as soft rice (or porridge); infant formulae; breakfast cereals; or the like. Optionally, one or more produced lipids can be incorporated into a dietary supplement, such as, for example, a vitamin or multivitamin. Optionally, an oil produced according to the method described herein can be included in a dietary supplement and optionally can be directly incorporated into a component of food or feed (e.g., a food supplement).

Examples of feedstuffs into which oils or lipids produced by the methods described herein can be incorporated include pet foods such as cat foods; dog foods; feeds for aquarium fish, cultured fish or crustaceans, etc.; or feed for farm-raised animals (including livestock and fish or crustaceans raised in aquaculture). Food or feed material into which the oils or lipids produced according to the methods described herein can be incorporated is preferably palatable to the organism which is the intended recipient. This food or feed material can have any physical properties currently known for a food material (e.g., solid, liquid, soft).

Optionally, one or more of the produced compounds (e.g., PUFAs) can be incorporated into a nutraceutical or pharmaceutical product. Examples of such a nutraceutical or pharmaceutical forms include various types of tablets, capsules, drinkable agents, etc. Optionally, the nutraceutical or pharmaceutical is suitable for topical application (e.g., lotion form). Dosage forms can include, for example, capsules, oils, tablets or the like.

The oil or lipids produced according to the methods described herein can be incorporated into products in combination with any of a variety of other agents. For instance, such compounds can be combined with one or more binders or fillers, chelating agents, pigments, salts, surfactants, moisturizers, viscosity modifiers, thickeners, emollients, fragrances, preservatives, etc., or any combination thereof.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein, and are not intended to limit the scope of the claims.

### EXAMPLES

### Example 1. Fatty Acid Synthesis Inhibitor Dose Timing Strategy

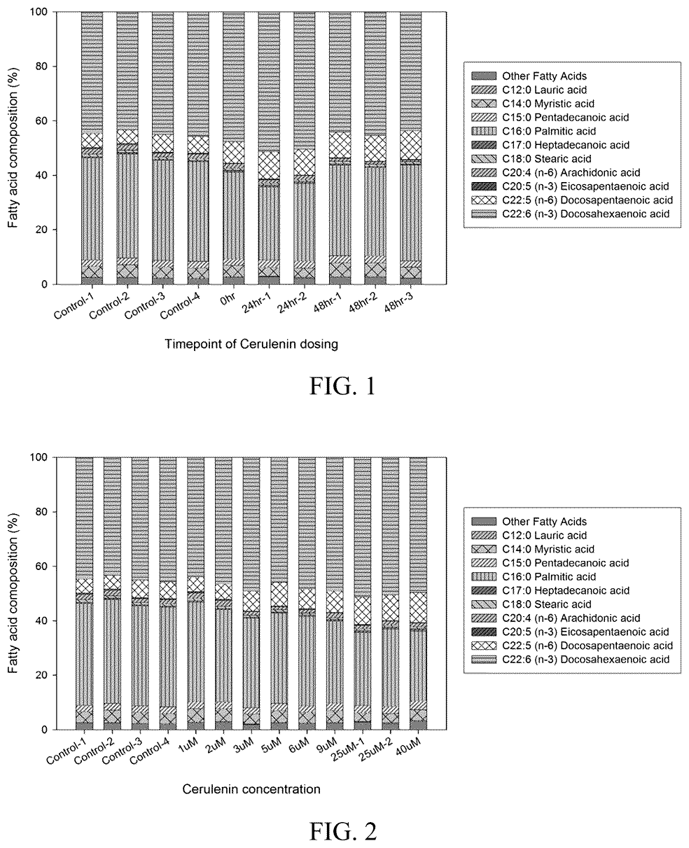

To determine the ideal time for addition of the fatty acid synthase inhibitor cerulenin without excessive harm such as preventing cell growth, experiments were conducted by adding a set amount of cerulenin at various amounts of time after inoculation. In all experiments, the control experiment was performed with the same medium and growth conditions without the addition of FAS inhibitors. The results demonstrated that cell growth was not inhibited at any addition time, however it did have a significant impact on the fatty acid profile (FIG. 1). Table 2 shows that adding cerulenin 24 hours after inoculation resulted in higher DHA and DPA content (% of TFA) with fatty acid synthesis pathway products C14:0 and C16:0 reduced by 21.1% and 27.9% respectively.

TABLE 1
Fatty acid content (in mg/g) of predominant fatty acids in Aurantiochytrium sp.
(G3) at 20° C. (except 48 hr). Addition times represent the time of addition of
25 μM cerulenin after inoculation.
Fatty Acid Content (mg/g of dry biomass)

| Time of | | | | C20:5 | C22:5 | C22:6 | | | DHA: |
|---|---|---|---|---|---|---|---|---|---|
| addition | C14:0 | C16:0 | C17:0 | (n-3) | (n-6) | (n-3) | SFA | TFA | DPA |
| Control | 25.50 | 230.24 | 3.07 | 3.10 | 35.26 | 277.28 | 280.75 | 619.90 | 7.98 |
| 0 hr | 23.45 | 183.73 | 4.07 | 1.97 | 45.07 | 276.37 | 232.31 | 579.14 | 6.13 |
| 24 hr | 18.23 | 150.17 | 4.36 | 1.90 | 55.95 | 287.63 | 204.48 | 560.24 | 5.14 |
| 48 hr* | 29.55 | 216.10 | 2.79 | 1.97 | 62.60 | 284.99 | 272.20 | 641.86 | 4.58 |

*From 25° C. data

TABLE 2
Fatty acid content (relative to total fatty acids) of predominant fatty
acids in Aurantiochytrium sp. (G3) at 20 °C. (except 48 hr).
Addition times represent the time of addition of 25 μM
cerulenin after inoculation
Fatty Acid Content (% of TFA)

| Time of | | | | C20:5 | C22:5 | C22:6 | |
|---|---|---|---|---|---|---|---|
| addition | C14:0 | C16:0 | C17:0 | (n-3) | (n-6) | (n-3) | SFA |
| Control | 4.12 | 37.16 | 0.50 | 0.50 | 5.67 | 44.71 | 45.32 |
| 0 hr | 4.05 | 31.73 | 0.70 | 0.34 | 7.78 | 47.72 | 40.11 |
| 24 hr | 3.25 | 26.80 | 0.76 | 0.33 | 9.99 | 51.34 | 35.35 |
| 48 hr* | 4.62 | 33.62 | 0.44 | 0.31 | 9.73 | 44.42 | 42.39 |

*From 25° C. data

### Example 2. Fatty Acid Synthesis Inhibitor Concentration

To determine the optimum concentration of cerulenin to provide the best results, a range of concentrations from 1 μM to 40 μM were tested. For consistency, all cerulenin additions were made at 24 hours after inoculation. FIG. 2 shows the fatty acid profiles of Aurantiochytrium sp. (G3) in varying concentrations of cerulenin at 20° C. Table 5 shows that increasing cerulenin concentration has a clear impact on the amount of C16:0 and DPA accumulated in the cells. In terms of percentage of the total fatty acids, those changes in specific fatty acids correspond to an increase in DHA content (% of TFA) from 44.71% to as high as 50.92% when 25 μM of cerulenin is used (Table 4).

TABLE 3
Fatty acid content (mg/g) of predominant fatty acids in Aurantiochytrium sp.
(G3) at 20° C. with cerulenin added at 24 hours after inoculation.

| Fatty Acid Content (mg/g of dry biomass) | | | | | | | | | |
|---|---|---|---|---|---|---|---|---|---|
| | | | | C20:5 | C22:5 | C22:6 | | | DHA: |
| Cerulenin | C14:0 | C16:0 | C17:0 | (n-3) | (n-6) | (n-3) | SFA | TFA | DPA |
| Control | 25.50 | 230.24 | 2.93 | 2.69 | 35.26 | 277.28 | 280.75 | 619.90 | 7.98 |
| 1 μM | 28.38 | 211.50 | 3.26 | 3.60 | 29.32 | 256.10 | 265.63 | 580.28 | 8.73 |
| 2 μM | 26.95 | 196.70 | 3.15 | 3.58 | 31.82 | 270.39 | 248.72 | 580.90 | 8.50 |
| 3 μM | 24.49 | 216.49 | 2.95 | 2.15 | 47.79 | 323.48 | 265.09 | 658.72 | 6.77 |
| 6 μM | 24.72 | 204.08 | 3.22 | 2.74 | 43.70 | 300.35 | 252.79 | 619.88 | 6.87 |
| 9 μM | 25.94 | 181.97 | 3.48 | 2.49 | 47.27 | 296.00 | 233.91 | 603.79 | 6.26 |
| 25 μM | 19.16 | 160.27 | 4.36 | 1.90 | 55.84 | 294.31 | 204.48 | 578.10 | 5.27 |
| 40 μM | 21.71 | 138.29 | 4.94 | 1.87 | 59.05 | 268.00 | 188.40 | 540.11 | 4.54 |

TABLE 4
Fatty acid content (relative to total fatty acids) of predominant
fatty acids in Aurantiochytrium sp. (G3) at 20° C. with
cerulenin added at 24 hours after inoculation.
Fatty Acid Content (% of TFA)

| | | | | C20:5 | C22:5 | C22:6 | |
|---|---|---|---|---|---|---|---|
| Cerulenin | C14:0 | C16:0 | C17:0 | (n-3) | (n-6) | (n-3) | SFA |
| Control | 4.12 | 37.16 | 0.50 | 0.50 | 5.67 | 44.71 | 45.32 |
| 1 μM | 4.89 | 36.45 | 0.56 | 0.62 | 5.05 | 44.13 | 45.78 |
| 2 μM | 4.64 | 33.86 | 0.54 | 0.62 | 5.48 | 46.55 | 42.82 |
| 3 μM | 3.72 | 32.87 | 0.45 | 0.33 | 7.26 | 49.11 | 40.24 |
| 6 μM | 3.99 | 32.92 | 0.52 | 0.44 | 7.05 | 48.45 | 40.78 |
| 9 μM | 4.30 | 30.14 | 0.58 | 0.41 | 7.83 | 49.02 | 38.74 |
| 25 μM | 3.31 | 27.70 | 0.76 | 0.33 | 9.67 | 50.92 | 35.35 |
| 40 μM | 4.02 | 25.60 | 0.91 | 0.35 | 10.93 | 49.62 | 34.88 |

### Example 3. Fatty Acid Synthesis Inhibitor Dosing Regimen

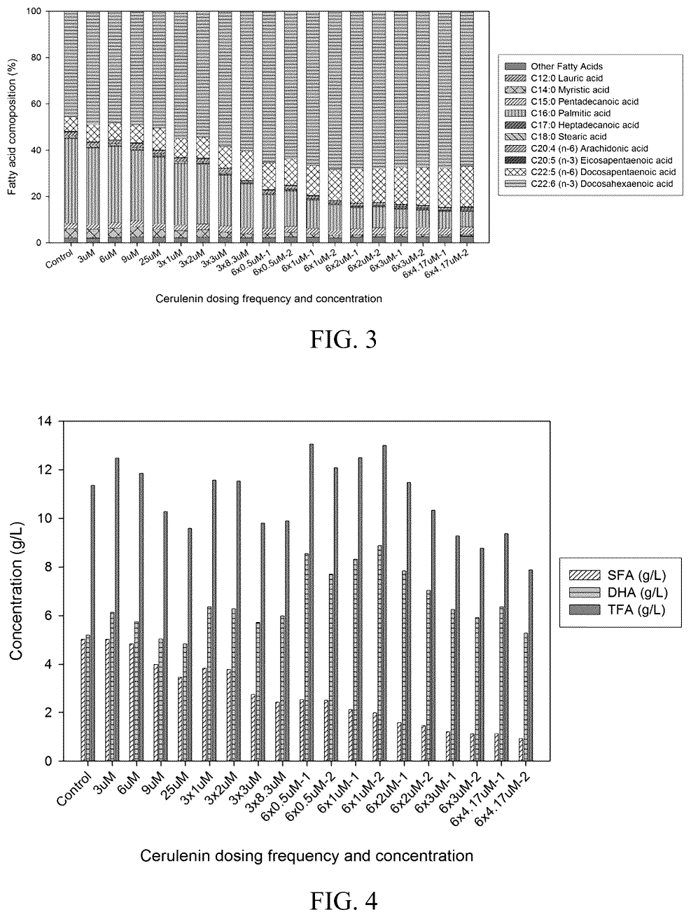

To investigate the effect of prolonged, repeated exposure to cerulenin, doses were spread equally across 3 or 6 times during the experiment. In each case, the total amount added was equivalent to the corresponding treatments that were added entirely at 24 hours. All additions began at 24 hours after inoculation. In FIGS. 3 and 4, a clear trend of increasing DHA and DPA was visible with both increasing cerulenin concentration and by dosing the cerulenin in multiple equivalent pulses. Furthermore, Table 5 shows no negative impact on total fatty acids when repeated dose addition is employed, while the actual amount of DHA (in mg/g) was increasing with increasing cerulenin. The highest DHA observed was using 6 equivalent doses of cerulenin, amounting to 12 μM in total, resulting in a DHA increase of 51.6%. There was also a maximal decrease in C14:0 and C16:0 of 85.1% and 83.9%, suggesting very effective inhibition of the FAS pathway by cerulenin under these conditions.

TABLE 5
Fatty acid content (in mg/g) of Aurantiochytrium sp. (G3) in various cerulenin
dosing strategies at 20° C. Treatments indicating 3× and 6× are multiple
additions of cerulenin at equally-spaced time intervals starting at 24 hours
after inoculation. For the 3× addition experiment, the interval was 24 hours;
while for the 6× addition experiment, the interval was 12 hours.

| Fatty Acid Content (mg/g of dry biomass) | | | | | | | | | |
|---|---|---|---|---|---|---|---|---|---|
| | | | | C20:5 | C22:5 | C22:6 | | | DHA: |
| Cerulenin | C14:0 | C16:0 | C17:0 | (n-3) | (n-6) | (n-3) | SFA | TFA | DPA |
| Control | 25.50 | 230.24 | 2.93 | 2.69 | 35.26 | 277.28 | 281.54 | 619.90 | 7.98 |
| 3 uM | 24.49 | 216.49 | 2.95 | 2.15 | 47.79 | 323.48 | 265.09 | 658.72 | 6.77 |
| 6 uM | 24.72 | 204.08 | 3.22 | 2.74 | 43.70 | 300.35 | 252.79 | 619.88 | 6.87 |
| 9 uM | 25.94 | 181.97 | 3.48 | 2.49 | 47.27 | 296.00 | 233.91 | 603.79 | 6.26 |
| 25 uM | 20.09 | 170.38 | 4.36 | 1.90 | 55.73 | 301.00 | 204.48 | 595.96 | 5.40 |
| 3 × 1 uM | 19.42 | 167.92 | 2.66 | 2.35 | 52.40 | 349.67 | 210.60 | 636.22 | 6.67 |
| 3 × 2 uM | 19.58 | 157.98 | 3.14 | 2.54 | 54.90 | 334.67 | 201.30 | 614.40 | 6.10 |
| 3 × 3 uM | 13.28 | 131.13 | 3.40 | 2.34 | 55.54 | 349.04 | 168.66 | 597.67 | 6.28 |
| 3 × 8.3 uM | 9.92 | 117.45 | 3.87 | 1.55 | 78.58 | 373.12 | 151.53 | 617.52 | 4.75 |
| 6 × 0.5 uM | 10.99 | 90.92 | 3.28 | 2.43 | 70.48 | 398.73 | 124.07 | 616.79 | 5.66 |
| 6 × 1 uM | 7.52 | 70.33 | 3.50 | 2.19 | 81.79 | 418.90 | 99.59 | 620.75 | 5.12 |
| 6 × 2 uM | 5.52 | 53.01 | 3.94 | 1.89 | 85.07 | 398.90 | 81.59 | 585.23 | 4.69 |
| 6 × 3 uM | 4.52 | 41.96 | 3.96 | 1.63 | 87.15 | 364.99 | 69.39 | 540.81 | 4.19 |
| 6 × 4.17 uM | 3.79 | 37.13 | 4.14 | 1.56 | 92.73 | 363.03 | 64.00 | 538.56 | 3.91 |

TABLE 6
Fatty acid content (relative to total fatty acids) of Aurantiochytrium sp. (G3) in various
cerulenin dosing strategies at 20° C. Treatments indicating 3× and 6× are multiple
additions of cerulenin at equally-spaced time intervals starting at 24 hours
after inoculation. For the 3× addition experiment, the interval was 24 hours;
while for the 6× addition experiment, the interval was 12 hours.
Fatty Acid Content (% of TFA)

| | | | | C20:5 | C22:5 | C22:6 | |
|---|---|---|---|---|---|---|---|
| Cerulenin | C14:0 | C16:0 | C17:0 | (n-3) | (n-6) | (n-3) | SFA |
| Control | 4.12 | 37.16 | 0.50 | 0.50 | 5.67 | 44.71 | 45.32 |
| 3 uM | 3.72 | 32.87 | 0.45 | 0.33 | 7.26 | 49.11 | 40.24 |
| 6 uM | 3.99 | 32.92 | 0.52 | 0.44 | 7.05 | 48.45 | 40.78 |
| 9 uM | 4.30 | 30.14 | 0.58 | 0.41 | 7.83 | 49.02 | 38.74 |
| 25 uM | 3.37 | 28.59 | 0.76 | 0.33 | 9.35 | 50.51 | 35.35 |
| 3 × 1 uM | 3.05 | 26.39 | 0.42 | 0.37 | 8.24 | 54.96 | 33.10 |
| 3 × 2 uM | 3.19 | 25.71 | 0.51 | 0.41 | 8.93 | 54.47 | 32.76 |
| 3 × 3 uM | 2.22 | 21.94 | 0.57 | 0.39 | 9.29 | 58.40 | 28.22 |
| 3 × 8.3 uM | 1.61 | 19.02 | 0.63 | 0.25 | 12.72 | 60.42 | 24.54 |
| 6 × 0.5 uM | 1.78 | 14.75 | 0.53 | 0.40 | 11.42 | 64.63 | 20.13 |
| 6 × 1 uM | 1.21 | 11.34 | 0.56 | 0.35 | 13.17 | 67.47 | 16.06 |
| 6 × 2 uM | 0.94 | 9.06 | 0.67 | 0.32 | 14.53 | 68.16 | 13.95 |
| 6 × 3 uM | 0.84 | 7.76 | 0.73 | 0.30 | 16.11 | 67.49 | 12.82 |
| 6 × 4.17 uM | 0.70 | 6.88 | 0.77 | 0.29 | 17.23 | 67.39 | 11.88 |

### Example 4. Fatty Acid Synthesis Inhibitor Effect on Product Yield

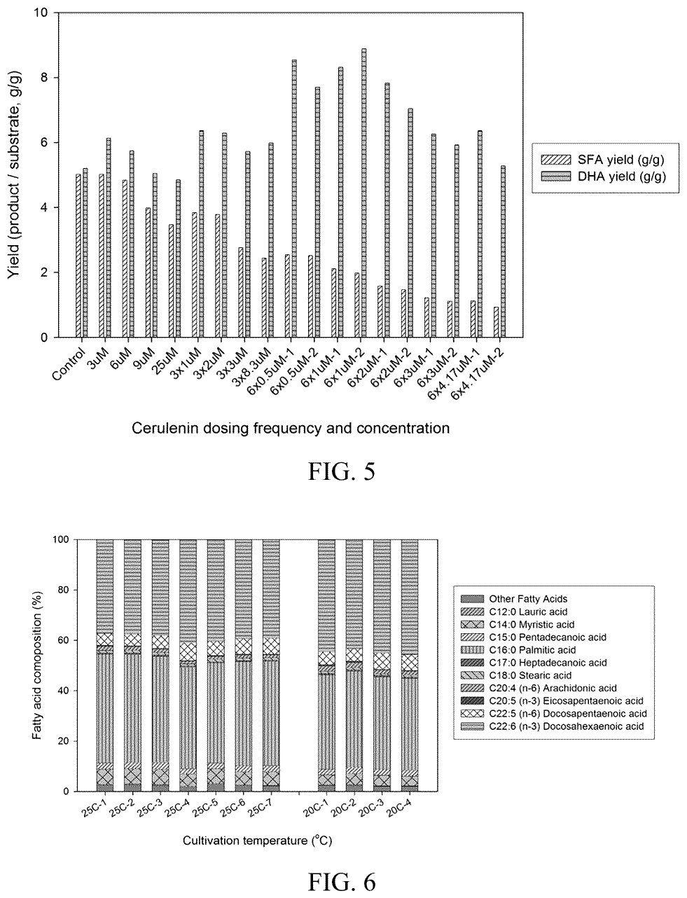

To assess the impact of the herein described methods for PUFA enhancement strategy, the yields of product (i.e. TFA, DHA or DPA) relative to the amount of carbon assimilated by the G3 cells was investigated. In FIG. 5, the total amount of product synthesized (categorized as total fatty acids (TFA), saturated fatty acids (SFA) or DHA) is depicted. In the presence of fatty acid synthesis inhibitors, dosed at certain frequency and concentration, both TFA and DHA concentration increased while SFA concentration decreased. For example, under the condition where cerulenin was dosed 1 μM each time for 6 times at 12 hour intervals, TFA and DHA increased by 15% and 71%, respectively, when compared to the results of the control experiment (FIG. 5). When the results were calculated as fatty acid yield (gram product/gram carbon consumed), DHA yield generally increased significantly while SFA yield reduced, under any cerulenin dosing conditions. For example, one of the optimal condition at 6×1 μM saw 53% increase in DHA yield and 64% reduction in SFA yield, when compared to the result of the control experiment, thereby suggesting that the cells were not only experiencing inhibition to the FAS pathway but they were up-regulating the PUFA synthase pathway to more efficiently utilize the available carbon.

### Example 5. Effects of Temperature on DHA % from Aurantiochytrium sp. (G3)

Flask scale experiments were executed to investigate the impact of decreasing the cultivation temperature (from 25° C. to 20° C.) on the fatty acid profile and the productivity of Aurantiochytrium sp. (G3). The data in FIG. 6 illustrate the increasing proportion of DHA and DPA with a decreased culture temperature to 20° C. Tables 7 and 8 show summary data exhibiting an increase in DHA of 14.7% paired with a decrease in C14:0 and C16:0 by 27.5% and 10.6%, respectively.

TABLE 7
Effect of different temperatures on predominant fatty acids (in mg/g) and their
relationships present in Aurantiochytrium sp. (G3)
Fatty Acid Content (mg/g of dry biomass)

| | | | | C20:5 | C22:5 | C22:6 | | | DHA: |
|---|---|---|---|---|---|---|---|---|---|
| | C14:0 | C16:0 | C17:0 | (n-3) | (n-6) | (n-3) | SFA | TFA | DPA |
| 25° C. | 35.73 | 262.05 | 2.58 | 2.59 | 35.68 | 246.18 | 324.31 | 630.78 | 7.03 |
| 20° C. | 25.50 | 230.24 | 3.07 | 3.10 | 35.26 | 277.28 | 280.75 | 619.90 | 7.98 |

TABLE 8
Effect of different temperatures on predominant fatty acids
(relative to total fatty acids) present in Aurantiochytrium sp. (G3).
Fatty Acid Content (% of TFA)

| | | | | C20:5 | C22:5 | C22:6 | |
|---|---|---|---|---|---|---|---|
| | C14:0 | C16:0 | C17:0 | (n-3) | (n-6) | (n-3) | SFA |
| 25° C. | 5.68 | 41.57 | 0.41 | 0.41 | 5.64 | 38.99 | 51.46 |
| 20° C. | 4.12 | 37.16 | 0.50 | 0.50 | 5.67 | 44.71 | 45.32 |

### Example 6. Effects of Full-Length Fermentation on DHA % from Aurantiochytrium sp. (G3)

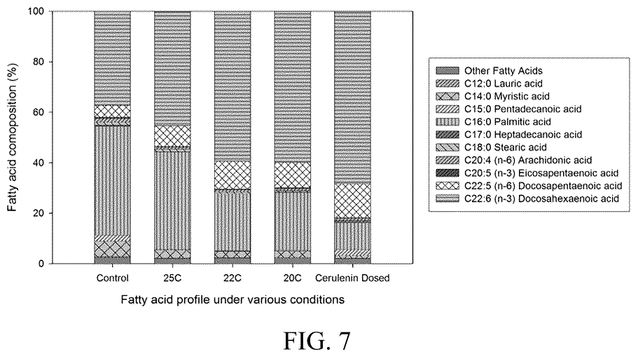

Full-length fermentations of Aurantiochytrium sp. G3 were carried out using 30 L stainless steel fermentors at varying temperatures (from 25° C. to 20° C.). These fermentations generally lasted from 150 hours to 200 hours with biomass and TFA reaching over 130 g/L and 55%, respectively. As can be seen in FIG. 7, full-length fermentation at a typical temperature of 25° C. was able to improve final DHA content over that from a typical control cultivation using flask; while full-length fermentations at lower temperatures were able to reach even higher DHA %. In the examples included in FIG. 7, fermentation at 25° C. reached 45% DHA, about 8% higher than that of a flask control at 37%. Fermentations at 22° C. and 20° C. achieved further DHA % increase at 59.7% and 60.0%, respectively. However, DHA content from the flask experiment using multiple and intermittent dosing of cerulenin was still the highest DHA % reached, at 68.7%.

### Example 7. Effects of Elevated Temperature on Aurantiochytrium sp. (G3)

G3 strains were cultured in 30-L fermenters for enhanced biomass and fatty acid production at 22° C., 25° C., and 28° C. G3 was pre-cultured in four Erlenmeyer flasks containing 500 mL of basal media (50 g/L glucose, 6.25 g/L yeast extract, 4 g/L MgSO₄·7H₂0, 4 g/L, 2.5 g/L NaCl, 2 mg/L copper sulfate, 2 mg/L zinc sulfate, 1 mg/L sodium molybdate, 1 mg/L cobalt (II) chloride, 1 mg/L manganese chloride and 1 mg/L nickel sulfate). Flasks were incubated under agitation at 25° C. and 200 rpm for 2 days. After the incubation period, three of the flasks (1.5 L) were used to inoculate 20 L of media in a 30 L bioreactor containing 175 g/L glucose, 9.66 g/L yeast extract, 2.57 g/L MgSO₄·7H₂0, 0.45 g/L sodium chloride, 6.44 g/L ammonium sulfate, 1.6 g/L potassium phosphate monobasic, 1.74 g/L potassium phosphate dibasic, 12.87 g/L monosodium glutamate, 0.1 g/L calcium chloride dehydrate, 1 mg/L copper sulfate, 1 mg/L zinc sulfate, 0.5 mg/L sodium molybdate, 0.5 mg/L cobalt (II) chloride, 0.5 mg/L manganese chloride, 0.5 mg/L nickel sulfate, 0.03 mg/L vitamin B12, 0.03 mg/L biotin and 6 mg/L thiamin hydrochloride and cultured in 30-L fermenters under the conditions of 22° C., 25° C., and 28° C. Agitation started at 325 rpm and increased to 365 rpm, aeriation was maintained at 0.3 vvm with atmospheric air, and pH 6.0. pH was maintained by the addition of base (27% NH₄OH). Vessels were fed to maintain a glucose consumption rate of approximately 3 g/L/h with a 750 g/L glucose solution. Cells were collected at various intervals and the biomass, TFA and lipid profiles were measured. Characteristics of the final G3 profiles at 25 and 28° C. are shown in Table 9. The lipid profiles are shown in Tables 10, 11, and 12.

TABLE 9
G3 Final Results at 22, 25 and 28° C.

| | G3 at 22° C. | G3 at 25° C. | G3 at 28° C. |
|---|---|---|---|
| Time, h | 186.89 | 140.19 | 165.67 |
| Biomass, g/L | 121.83 | 107.92 | 104.16 |
| TFA, % biomass | 49.6 | 56.3 | 59.2 |
| DHA, % TFA | 66.78 | 64.1 | 58.6 |
| SFA, % TFA | 16.18 | 19 | 25 |
| MUFA, % TFA | | 0.9 | 0.8 |

TABLE 10
Fatty Acid Profile of G3 Cultured at 22° C.

| | | | | | | | | | | | 20:5 | | 22:5 | C22:5 | C22:6 |
|---|---|---|---|---|---|---|---|---|---|---|---|---|---|---|---|
| | | | | | | | | 18:1 | | 20:4 | (n-3) | | (n-6) | (n-3) | (n-3) |
| Lipid: | 12:0 | 14:0 | 14:1 | 15:0 | 16:0 | 16:1 | 18:0 | Vac | 20:0 | (n-6) | EPA | 22:0 | DPA | DPA* | DHA* |
| 45 h | 0.06 | 0.96 | 0.24 | 0.34 | 24.64 | 0.16 | 1.10 | 0.32 | 0.12 | 0.65 | 0.50 | 0.24 | 9.44 | 0.24 | 58.47 |
| 68 h | 0.08 | 1.35 | 0.29 | 0.24 | 21.17 | 0.23 | 0.86 | 0.39 | 0.14 | 0.65 | 0.38 | 0.16 | 10.25 | 0.16 | 61.51 |
| 90 h | 0.08 | 1.30 | 0.47 | 0.20 | 17.12 | 0.32 | 0.68 | 0.38 | 0.17 | 0.89 | 0.39 | 0.17 | 10.61 | 0.13 | 65.25 |
| 115 h | 0.08 | 1.17 | 0.63 | 0.12 | 14.17 | 0.38 | 0.53 | 0.37 | 0.16 | 1.08 | 0.42 | 0.14 | 10.91 | 0.12 | 68.07 |
| 141 h | 0.08 | 1.06 | 0.71 | 0.10 | 13.21 | 0.41 | 0.49 | 0.40 | 0.15 | 1.15 | 0.44 | 0.14 | 11.57 | 0.13 | 68.22 |
| 163 h | 0.08 | 1.11 | 0.66 | 0.10 | 13.71 | 0.40 | 0.51 | 0.41 | 0.15 | 1.08 | 0.46 | 0.14 | 11.80 | 0.14 | 67.50 |
| 187 h | 0.08 | 1.11 | 0.62 | 0.09 | 14.32 | 0.37 | 0.52 | 0.43 | 0.15 | 1.02 | 0.47 | 0.15 | 12.02 | 0.15 | 66.69 |

TABLE 11
Fatty Acid Profile of G3 Cultured at 25° C.

| | | | | | | | | | | | 20:5 | | 22:5 | C22:5 | C22:6 |
|---|---|---|---|---|---|---|---|---|---|---|---|---|---|---|---|
| | | | | | | | | 18:1 | | 20:4 | (n-3) | | (n-6) | (n-3) | (n-3) |
| Lipid: | 12:0 | 14:0 | 14:1 | 15:0 | 16:0 | 16:1 | 18:0 | Vac | 20:0 | (n-6) | EPA | 22:0 | DPA | DPA* | DHA* |
| 63 h | 0.11 | 1.07 | 0.23 | 0.21 | 20.93 | 0.16 | 0.83 | 0.17 | 0.14 | 0.54 | 0.33 | 0.12 | 12.02 | 0.16 | 60.79 |
| 92 h | 0.15 | 1.29 | 0.35 | 0.17 | 19.66 | 0.22 | 0.78 | 0.19 | 0.16 | 0.62 | 0.29 | 0.17 | 12.00 | 0.12 | 60.78 |
| 116 h | 0.08 | 1.19 | 0.49 | 0.12 | 16.18 | 0.25 | 0.59 | 0.19 | 0.15 | 0.81 | 0.33 | 0.12 | 12.94 | 0.12 | 64.60 |
| 140 h | 0.08 | 1.22 | 0.43 | 0.11 | 16.85 | 0.25 | 0.56 | 0.21 | 0.13 | 0.75 | 0.35 | 0.10 | 12.99 | 0.14 | 64.13 |

TABLE 12
Fatty Acid Profile of G3 Cultured at 28° C.

| | | | | | | | | | | | 20:5 | | 22:5 | C22:5 | C22:6 |
|---|---|---|---|---|---|---|---|---|---|---|---|---|---|---|---|
| | | | | | | | | 18:1 | | 20:4 | (n-3) | | (n-6) | (n-3) | (n-3) |
| Lipid | 12:0 | 14:0 | 14:1* | 15:0 | 16:0 | 16:1 | 18:0 | Vac | 20:0 | (n-6) | EPA | 22:0 | DPA | DPA* | DHA* |
| 63 h | 0.10 | 1.24 | 0.22 | 0.21 | 19.28 | 0.16 | 0.71 | 0.13 | 0.13 | 0.50 | 0.32 | 0.12 | 13.66 | 0.19 | 60.76 |
| 92 h | 0.12 | 1.26 | 0.26 | 0.17 | 17.29 | 0.18 | 0.61 | 0.14 | 0.15 | 0.49 | 0.28 | 0.12 | 14.47 | 0.20 | 62.22 |
| 116 h | 0.13 | 1.41 | 0.40 | 0.16 | 18.75 | 0.23 | 0.65 | 0.20 | 0.15 | 0.68 | 0.30 | 0.11 | 13.67 | 0.16 | 61.10 |
| 140 h | 0.08 | 1.60 | 0.39 | 0.15 | 21.05 | 0.25 | 0.67 | 0.23 | 0.12 | 0.74 | 0.33 | 0.10 | 13.02 | 0.15 | 59.36 |
| 166 | 0.09 | 1.66 | 0.31 | 0.14 | 22.10 | 0.23 | 0.66 | 0.22 | 0.10 | 0.75 | 0.44 | 0.08 | 12.84 | 0.16 | 58.55 |

What is claimed is:

1. A microbial oil comprising at least 85% total fatty acids by weight, wherein the total fatty acids comprise at least 50% docosahexaenoic acid by weight, between 6% to 18% docosapentaenoic acid n-6 by weight, and less than 1% stearic acid by weight.

2. A microbial oil comprising at least 85% total fatty acids by weight, wherein the total fatty acids comprise one or more of at least 60% docosahexaenoic acid by weight, 0.01% to 1% stearic acid by weight, 10% to 18% docosapentaenoic acid n-6 by weight, 4% myristic acid by weight, less than 30% palmitic acid by weight, and 0.1% to 0.5% heptadecanoic acid by weight.

3. The microbial oil of claim 1, wherein the total fatty acids comprise one or more of less than 1% eicosapentaenoic acid by weight and less than 3% pentadecanoic acid by weight.

4. The microbial oil of claim 1, wherein the total fatty acids comprise less than 40% saturated fatty acids.

5. The microbial oil of claim 1, wherein the total fatty acids comprise one or more of 60% to 70% docosahexaenoic acid by weight, 0.01% to 2.0% arachidonic acid by weight, and 0.01% to 1% eicosatetraenoic acid by weight.

6. The microbial oil of claim 1, wherein the microbial oil comprises at least 90% triglycerides.

7. The microbial oil of claim 1, wherein the ratio of docosahexaenoic acid to docosapentaenoic acid n-6 is less than or equal to 7:1.

8. The microbial oil of claim 1, wherein the oil is produced by culturing an Aurantiochytrium microorganism in a culture medium at a temperature from 18° to 28° C.

9. A method of making a biomass comprising culturing an oil-producing Aurantiochytrium microorganism in a culture medium comprising a fatty acid synthesis inhibitor, wherein the biomass comprises at least 500 mg/g oil.

10. The method of claim 9, wherein the culture medium is at a temperature of 18° C. to 28° C.

11. The method of claim 9, wherein the method further comprises adding the microorganism to the culture medium and wherein the fatty acid synthesis inhibitor is added to the culture medium between 24 to 48 hours after addition of the microorganism to the culture medium.

12. The method of claim 9, wherein the method further comprises adding the microorganism to the culture medium and wherein the fatty acid synthesis inhibitor is added to the culture medium at least 6, 12, 24 or 48 hours after addition of the microorganism to the culture medium.

13. The method of claim 9, wherein the culturing comprises adding one or more doses of the fatty acid synthesis inhibitor to the culture medium.

14. The method of claim 9, wherein the method further comprises adding the microorganism to the culture medium and wherein the culturing comprises adding the fatty acid synthesis inhibitor in 2, 3, 4, 5, or 6 doses beginning 12 hours after addition of the microorganism to the culture medium.

15. The method of claim 14, wherein the doses are added every 6, 12, or 24 hours.

16. The method of claim 9, wherein a total concentration of the fatty acid synthesis inhibitor added to the culture medium during the culturing comprises between 3 and 40 μM.

17. The method of claim 9, wherein a total concentration of the fatty acid synthesis inhibitor added to the culture medium during the culturing comprises between 30 μg and 700 μg of inhibitor per gram of biomass.

18. The method of claim 9, wherein the fatty acid synthesis inhibitor is fed to the culture medium.

19. The method of claim 9, wherein the microorganism has IDAC Accession No. 220716-01.

20. The method of claim 9, wherein the method further comprises isolating the oil from the biomass.

21. The method of claim 9, wherein the oil comprises fatty acids and the fatty acids comprise at least 50% docosahexaenoic acid by weight, between 6% to 18% docosapentaenoic acid n-6 by weight, and less than 1% stearic acid by weight.

22. The method of claim 9, wherein the oil comprises fatty acids and the fatty acids comprise one or more of 10% to 18% docosapentaenoic acid n-6 by weight, less than 1% eicosapentaenoic acid by weight, 10% to 40% saturated fatty acids, 50% to 70% docosahexaenoic acid by weight, and at least 95% by weight triglycerides, and 0.1% to 0.5% heptadecanoic acid by weight.

23. The method of claim 9, wherein the oil comprises fatty acids and the fatty acids comprise at least 60% docosahexaenoic acid by weight and between 10% by weight to 18% docosapentaenoic acid n-6 by weight.
